(19) United States
(12) Patent Application Publication
(10) Pub. No.: US 2018/0299117 A1
(43) Pub. Date: Oct. 18, 2018

(54) ILLUMINATION APPARATUS WITH AIR FILTRATION AND ULTRAVIOLET STERILIZATION FUNCTIONS

(71) Applicant: GUANGDONG XINGUANGYUAN ELECTRONICS CO., LTD, Dongguan City (CN)

(72) Inventor: HUA CHU MIN, Dongguan City (CN)

(21) Appl. No.: 15/488,634

(22) Filed: Apr. 17, 2017

(51) Int. Cl. F21V 33/00; A61L 9/20; F24F 3/16; F24F 13/06

(52) U.S. Cl. CPC F21V 33/0064; A61L 9/20; F24F 3/1603; F24F 2221/02; A61L 2209/14; F24F 2003/1682; F24F 2003/1667; F24F 13/06

(57) ABSTRACT

An illumination apparatus with air filtration and ultraviolet sterilization functions includes a cover, a carrier board, and a base. The cover includes a first region, a second region, a third region, a light source, and meshes. The light source is disposed at the second region. The meshes are disposed at the first region and the second region respectively. The carrier board is attached to the cover and includes a plate, a plurality of openings, an ultraviolet light, and a negative ionizer. The openings are formed on the plate for leading air flowing from the meshes. The ultraviolet light is disposed between the openings for sterilizing the flowing air. The negative ionizer is disposed on the plate to generate negative ions in the air. The base is attached to the carrier board and includes a ventilator for providing suction power to lead the flowing air.

### FIELD

The present disclosure relates to an illumination apparatus and more particularly to an illumination apparatus with air filtration and ultraviolet sterilization functions.

### BACKGROUND

This section provides background information related to the present disclosure which is not necessarily prior art.

The progress of modern society has brought people to live and work conveniently, but has also caused serious environmental pollution. Especially in densely populated areas, venues such as schools, hospitals, supermarkets, office buildings and others are prone to poor air quality and hence large-scale rapid virus or bacterial infection because of the confined space or large flows of people coming and going. Accordingly, there is an urgent need for a sterilization and filtration system that can be used in these situations to solve the problem.

### SUMMARY

This section provides a general summary of the disclosure, and is not a comprehensive disclosure of its full scope or all of its features.

An aspect of the present disclosure is to develop an illumination apparatus to include air filtration and ultraviolet sterilization functions.

In order to achieve the aforementioned purpose in the present disclosure, the present disclosure provides an illumination apparatus with air filtration and ultraviolet sterilization functions, and the illumination apparatus includes a cover, a carrier board and a base. The cover has a first region, a second region and a third region, and the second region is located between the first region and the third region. The cover includes a light source and a plurality of meshes. The light source is disposed at the second region. The meshes are disposed at the first region and the second region respectively. The carrier board is attached to the cover and includes a plate, a plurality of openings, an ultraviolet light and a negative ionizer. The openings are formed on the plate for leading air flowing from the meshes. The ultraviolet light is disposed on the plate and located between the openings for sterilizing the air flowing from the meshes. The negative ionizer is disposed on the plate to generate negative ions in the air. The base is attached to the carrier board and includes a ventilator for providing suction power to lead the air flowing within the illumination apparatus.

An advantage that may be realized by the present disclosure is to include the air filtration and sterilization functions embedded within the illumination apparatus without occupying too much space. Since the light source of the illumination apparatus is preferred to be an LED, the overall size of the illumination apparatus will not be enlarged too much when including air filtration and sterilization functions inside the illumination apparatus.

Other aspects, advantages, and novel features of the disclosure will become more apparent from the following detailed description when taken in conjunction with the accompanying drawings.

### DRAWINGS

The drawings described herein are for illustrative purposes only of selected embodiments and not all possible implementations, and are not intended to limit the scope of the present disclosure.

FIG. 1 is a perspective view of an illumination apparatus with air filtration and ultraviolet sterilization functions according to an exemplary embodiment of the present disclosure;

FIG. 2 is a partial exploded view of the illumination apparatus with air filtration and ultraviolet sterilization functions according to an exemplary embodiment of the present disclosure;

FIG. 3 is an exploded view of the illumination apparatus with air filtration and ultraviolet sterilization functions according to an exemplary embodiment of the present disclosure; and

FIG. 4 is a lateral view of the illumination apparatus with air filtration and ultraviolet sterilization functions according to an exemplary embodiment of the present disclosure.

### DETAILED DESCRIPTION

These and other aspects of the embodiments herein will be better appreciated and understood when considered in conjunction with the following description and the accompanying drawings.

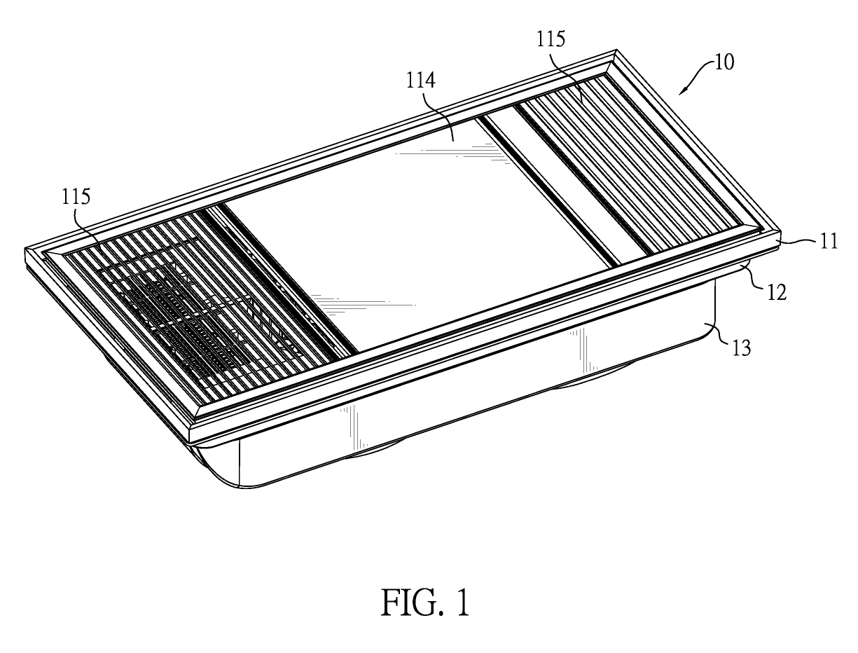

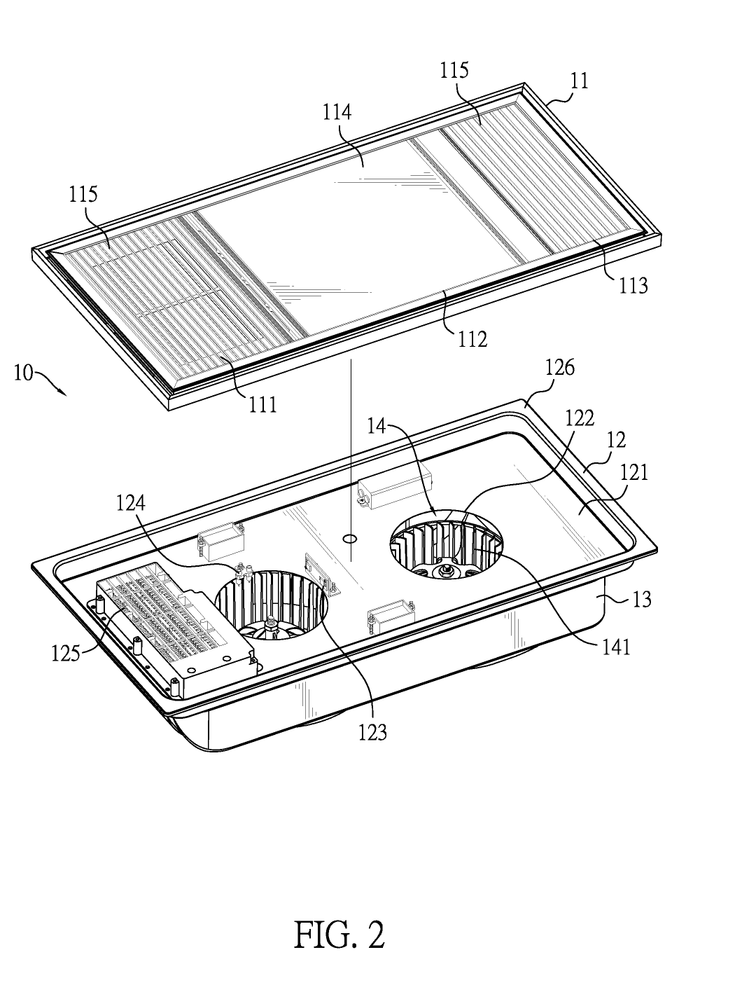

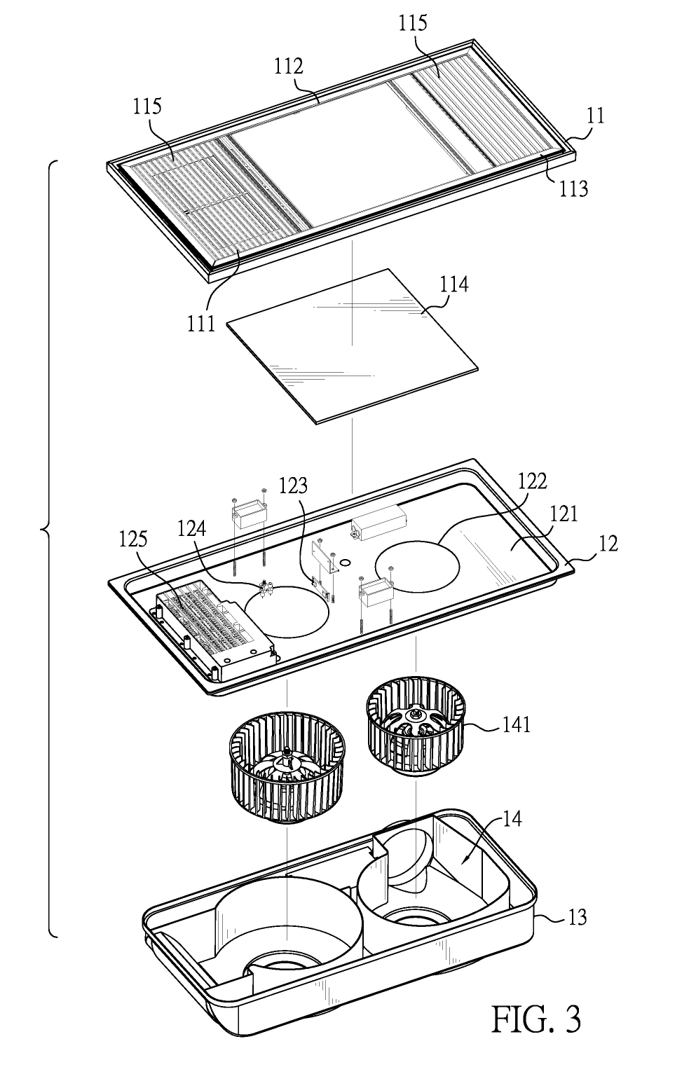

With reference to FIG. 1, FIG. 2 and FIG. 3, an illumination apparatus 10 with air filtration and ultraviolet (UV) sterilization functions includes a cover 11, a carrier board 12 and a base 13. The carrier board 12 is attached to the cover 11, and the base 13 is attached to the carrier board 12.

The cover 11 includes a first region 111, a second region 112 and a third region 113, a light source 114 and a plurality of meshes 115. The first region 111 and the third region 113 are located at two sides of the cover 11, and the first region 111 and the third regions 113 are inlets for air passing. The second region 112 is located between the first region 111 and the third region 113. The light source 114 is disposed in the second region 112. The meshes 115 are disposed in the first region 111 and the third region 113 respectively. The meshes 115 are detachable from the first region 111 and the third region 113 of the cover 11 respectively.

The carrier board 12 includes a plate 121, a plurality of openings 122, an ultraviolet light 123, a negative ionizer 124 and an air heater 125. The plate 121 is surrounded by a wall 126 and is configured to accommodate the ultraviolet light 123, the negative ionizer 124 and the air heater 125. The openings 122 are formed on the plate 121 for leading the air flowing from the meshes 115 at the first region 111 and the third region 113 into the illumination apparatus 10. The ultraviolet light 123 is disposed on the plate 121 and located between the openings 122 for sterilizing the air flowing from the meshes 115. The reason for disposing the ultraviolet light 123 within the illumination apparatus 10 is to prevent ultraviolet leakage.

The negative ionizer 124 is also disposed on the plate 121 and adjacent to one of the openings 122. The negative ionizer 124 is configured to provide negative ions in the air, which is sterilized by the ultraviolet light 123. The air heater 125 is disposed at one side of the plate 121 and is configured to heat the air to a selected temperature.

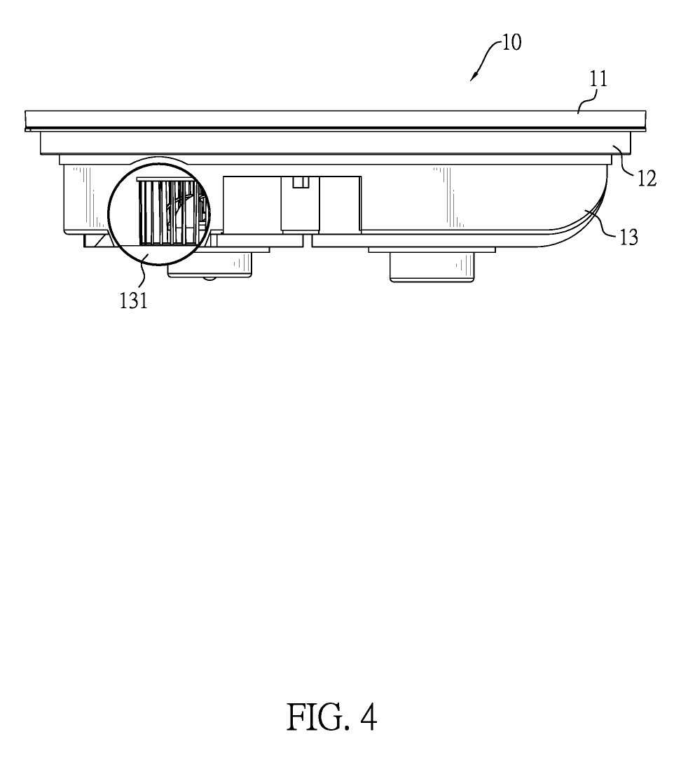

The base 13 includes a ventilator 14 and the ventilator 14 includes a plurality of fins 141. The ventilator 14 is preferred to be an air blower with low noise. The fins 141 are disposed on the base 13 and respectively corresponding to the openings 122 of the carrier board 12. The ventilator 14 is configured to provide suction power to lead the air flowing within the illumination apparatus. Specifically, the ventilator 14 leads the air flowing from the meshes 115 to the openings 122 of the carrier board 12. Then, the air will be sucked into the ventilator 14 and exhausted from a vent 131 at one lateral side of the base 13, as shown in FIG. 4.

The meshes 115 are preferred to be air filters to filter dust from the air. After filtering the dust from the air, the air flows into the carrier board 12 of the illumination apparatus 10. The ultraviolet light 123 within the carrier board 12 sterilizes the air to kill bacteria in the air. The negative ionizer 124 provides negative ions for the air. Furthermore, the illumination apparatus 10 also includes the air heater 125 to heat the air to a selected temperature.

The light source 114 is preferred to be made of light emitting diodes (LED) with advantages such as low light decay, no glare, ultra thinness and so on. The light source 114 may include different light colors, such as red, green, blue, warm white and pure white. The brightness and the colors of the light source 114 are adjusted and driven by Pulse Width Modulation (PWM) technique. The PWM to drive the LED light source is well known for a person with ordinary skill in the art and the detailed description of the function of PWM is omitted.

The illumination apparatus 10 may further include a Bluetooth adapter or an infrared-rays (IR) adapter, and the illumination apparatus 10 is capable of being remotely controlled by a Bluetooth controller or an IR controller.

The air filtration and sterilization functions are embedded within the illumination apparatus 10 without occupying too much space. Since the light source 112 of the illumination apparatus 10 is preferred to be an LED light source, the overall size of the illumination apparatus 10 will not be enlarged too much when including air filtration and sterilization functions inside the illumination apparatus. While the present disclosure has been described in terms of what are presently considered to be the most practical and preferred embodiments, it is to be understood that the present disclosure need not be restricted to the disclosed embodiment. On the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present disclosure which is defined by the appended claims.

What is claimed is:

1. An illumination apparatus with air filtration and ultraviolet sterilization functions, comprising:
a cover having a first region, a second region, and a third region, the second region located between the first region and the third region, and the cover including:
a light source disposed at the second region; and
a plurality of meshes disposed at the first region and the second region respectively;
a carrier board attached to the cover and including:
a plate;
a plurality of openings formed on the plate for guiding air flowing from the meshes;
an ultraviolet light disposed on the plate and located between the openings for sterilizing the air flowing from the meshes; and
a negative ionizer disposed on the plate to generate negative ions; and
a base attached to the carrier board and including a ventilator that provides suction power to lead the air flowing within the illumination apparatus.

2. The illumination apparatus with air filtration and ultraviolet sterilization functions as claimed in claim 1, wherein the plate of the carrier board is surrounded by a wall.

3. The illumination apparatus with air filtration and ultraviolet sterilization functions as claimed in claim 1, wherein the negative ionizer is adjacent to the openings.

4. The illumination apparatus with air filtration and ultraviolet sterilization functions as claimed in claim 1, wherein the base further includes a vent formed at a lateral side of the base.

5. The illumination apparatus with air filtration and ultraviolet sterilization functions as claimed in claim 1, wherein the light source is a light emitting diode (LED).

6. The illumination apparatus which air filtration and ultraviolet sterilization functions as claimed in claim 1, wherein the ventilator includes a plurality of fins, and the fins are disposed on the base and respectively corresponding to the openings of the carrier board.

7. The illumination apparatus which air filtration and ultraviolet sterilization functions as claimed in claim 1, wherein the ventilator is an air blower with low noise.

8. The illumination apparatus with air filtration and ultraviolet sterilization functions as claimed in claim 1, further comprising an air heater disposed at one side of the plate and configured to heat the air to a selected temperature.

9. The illumination apparatus with air filtration and ultraviolet sterilization functions as claimed in claim 8, wherein the plate of the carrier board is surrounded by a wall.

10. The illumination apparatus with air filtration and ultraviolet sterilization functions as claimed in claim 8, wherein the negative ionizer is adjacent to the openings.

11. The illumination apparatus with air filtration and ultraviolet sterilization functions as claimed in claim 8, wherein the base further includes a vent formed at a lateral side of the base.

12. The illumination apparatus with air filtration and ultraviolet sterilization functions as claimed in claim 8, wherein the light source is a light emitting diode (LED).
